(19) United States
(12) Patent Application Publication
(10) Pub. No.: US 2014/0046411 A1
(43) Pub. Date: Feb. 13, 2014

(54) Headliner Cooling System

(71) Applicant: Welkins, LLC, Roseville, CA (US)

(72) Inventors: William Elkins, Lincoln, CA (US); Theodore Maurice Jordan, JR., Elk Grove, CA (US); Christopher Crockett Moore, Loomis, CA (US); Christopher Blodgett, Roseville, CA (US)

(73) Assignee: Welkins, LLC, Roseville, CA (US)

(21) Appl. No.: 13/963,167

(22) Filed: Aug. 9, 2013

Related U.S. Application Data
(60) Provisional application No. 61/681,505, filed on Aug. 9, 2012.

(51) Int. Cl. A61F 7/00

(52) U.S. Cl. CPC A61F 7/00
USPC 607/104

(57) ABSTRACT

A cooling system for cooling a portion of an individual's body via a body-conformed apparatus. The cooling system includes a unit remote from the body and tethered to the body conformed apparatus. A replaceable ice cartridge is disposed in the unit and relative to a coolant pathway that circulates coolant to and from the individual. A control valve within the coolant pathway is operable to control the amount of coolant passing the ice cartridge and thus control the temperature of the coolant leaving the unit.

### RELATED APPLICATIONS

This application claims priority to, U.S. Provisional Patent Application No. 61/681,505 having a filing date of Aug. 9, 2012, which is incorporated herein by reference, in its entirety.

### FEDERALLY SPONSORED RESEARCH OR DEVELOPMENT

This invention was made with government support under DoD Contract HQ003410-C-0031 awarded by the Department of Defense and administered by WHS ACQUISITION & PROCUREMENT OFFICE for the Joint IED Defeat Organization (JIE DDO). The contract gives the government certain rights in the invention. However, the government later terminated the contract.

### MICROFICHE/COPYRIGHT REFERENCE

[Not Applicable]

### BACKGROUND OF THE INVENTION

The invention relates to heat transfer and cooling systems for cooling a portion of an individual's body via a body-conformed apparatus, and more particularly relates to a method and device for controlling the amount of temperature change caused to the portion of the individual's body for treating in situ various human injuries, for example, stroke, traumatic brain injury, cardiac arrest, and significant blood loss.

In treating and successfully recovering from head injury, time is critical. In particular, when brain cells die, the associated function provided by those brain cells is more apt to be lost by the patient. When a sufficient number of brain cells have died, such function is lost, and the potential for the function returning decreases as more and more brain cells die. Hence, it is imperative that therapy directed to the brain injury be executed as soon as possible after the brain injury occurs to minimize such deleterious effects.

An interesting phenomenon has been observed by those who study brain injury relating to the brain cell survival rate as a function of time when brain temperature is reduced. In particular, drowning victims in exceptionally cold water have in some cases been submerged and deprived of oxygen for tens of minutes. While such a time period would ordinarily cause such extensive brain cell loss that significant brain function loss would occur, it has been observed that brain function has in many cases been restored completely, or nearly completely. Based on these observations, it has been determined that by cooling the patient's head, an effect similar to “slowing down the clock” can be achieved. Accordingly, a need exists for extending this observed benefit from accidental occurrences to intentional use of this effect in the beneficial treatment of brain injury, and particularly stoke and head trauma.

### BRIEF SUMMARY OF THE INVENTION

It is therefore an object of the present invention to protect a patient's brain from further damage from a moment of injury until and during brain injury therapy.

Another object is to provide a cooling system which can be used to cool the patient or a particular potion of the patient's body shortly after brain or other body injury.

It is another object to provide a cooling system for use remotely from a medical facility.

It is another object to provide a cooling system for use within a medical facility.

It is another object to provide two cooling systems wherein the first cooling system may be interchanged during treatment of a patient with the second cooling system.

These and other objects of the invention are achieved in a cooling system for cooling a portion of an individual's body. The system includes a body conformed apparatus for attachment directly to the patient, a unit remote from the body and an umbilical tubing connecting the body conformed apparatus to the unit. A coolant flows from the unit to the body conformed apparatus via several pathways relative to a heat sink cooling source in order to regulate the temperature of the coolant and thus control the cooling of the body portion to which the body conformed apparatus is connected.

### BRIEF DESCRIPTION OF SEVERAL VIEWS OF THE DRAWINGS

FIG. 1 is a perspective view of an EMT unit cooling system and an ICU unit cooling system of the present invention.

FIG. 2 is a block diagram of the cooling system attached to a patient.

FIGS. 3A and 3B are perspective views of the headliner assembly of the cooling system of FIG. 1.

FIG. 4 is a flat plan view of a cap portion of the headliner assembly of FIGS. 3A and 3B.

FIG. 5 is a diagram illustration representing a cross section of the layers of the cap portion of FIG. 4.

FIG. 6 is a perspective view of the ice cartridge of FIG. 2.

FIGS. 7A-C are respective end, side and top views of the ice cartridge of FIG. 6.

FIG. 8 is a diagram illustration representing a cross section of the layers of the ice cartridge and cooling heat exchanger of FIG. 2.

FIG. 9 is a block diagram of a controller board and associated devices.

FIG. 10 is a graph diagram of patient temperature over time.

FIG. 11 is a diagram of a control display panel of the EMT unit of FIG. 1.

FIG. 12 is a diagram of a control display panel of the EMT unit of FIG. 1.

FIG. 13 is a diagram of a control display panel of the ICU unit of FIG. 1.

FIG. 14 is a diagram view of the components and pathway flows of the EMT unit of FIG. 1.

FIG. 15 is a diagram view of the components and pathway flows of the ICU unit of FIG. 1.

FIG. 16 is a side view of a two port connector of the cooling systems of FIG. 1.

FIG. 17 is a perspective transparent side view of the liquid pump of the cooling system of FIG. 1.

### DETAILED DESCRIPTION OF THE INVENTION

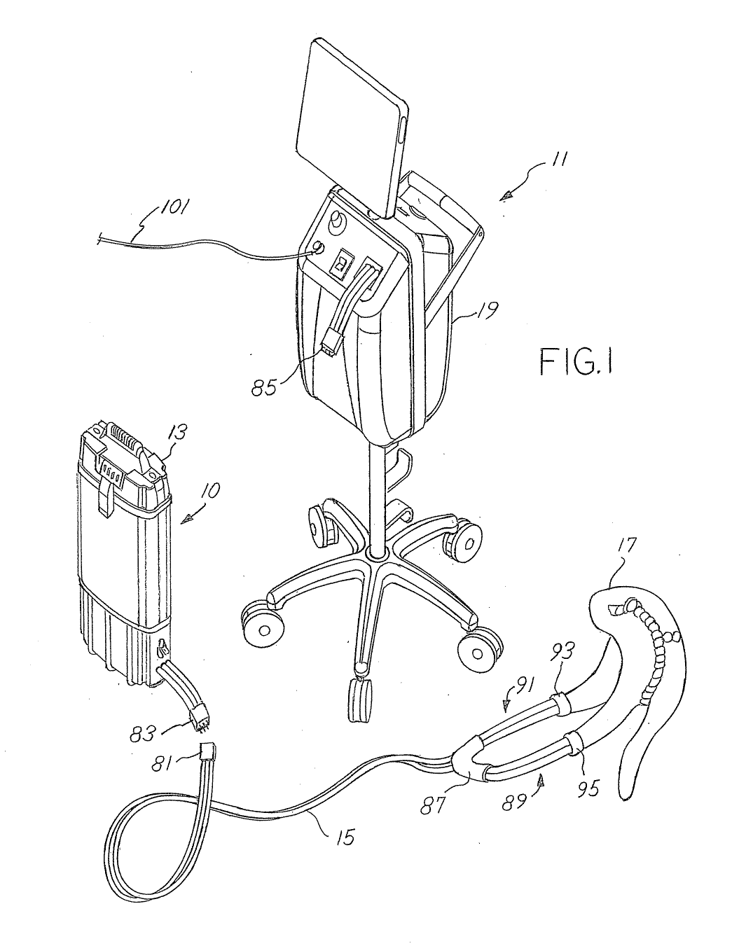

Referring to FIG. 1, two separate cooling systems 10 and 11 are shown. Cooling system 10 includes an EMT conditioning unit 13 (“Emergency Medical Team” conditioning unit), an umbilical tubing 15 and a body-conformal heat exchanger 17. Cooling system 11 includes an ICU conditioning unit 19 (“Intensive Care Unit” conditioning unit), umbilical tubing 15 and body conformal heat exchanger 17. In one embodiment, the EMT unit 13 may be constructed for ease of transport to a location where the patient has a need, for example, a medical emergency. The EMT unit and patient are transported to an intensive care facility where the ICU conditioning unit 19 is located and there used to continue the therapy; the EMT unit is disconnected from tubing 15 and the ICU unit is attached to the same tubing 15. In other embodiments, the EMT unit 13 may be used in other applications such as home use, athletic side line cooling, etc.

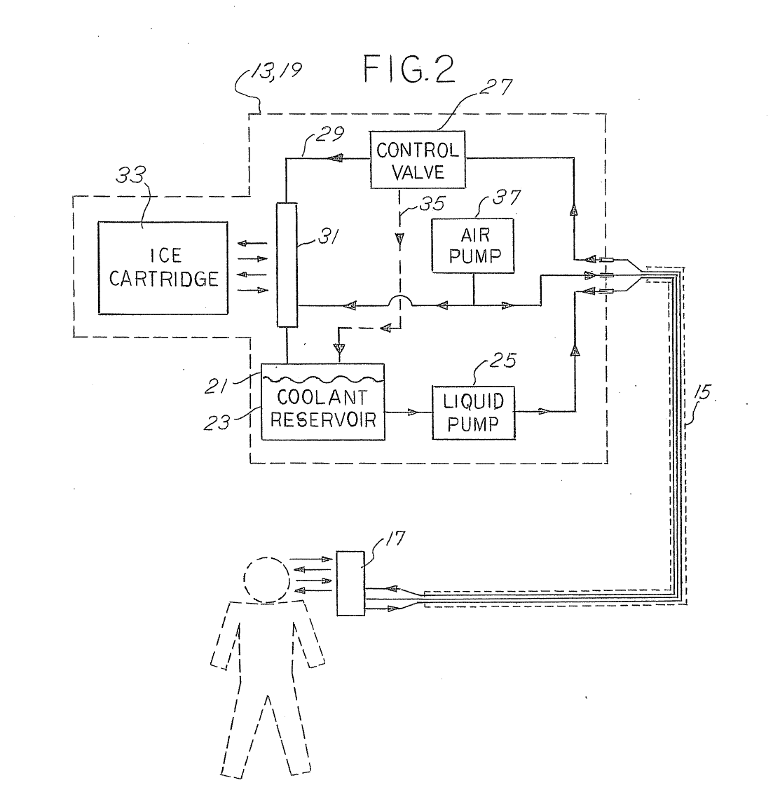

Operation of cooling systems 11, 13 is illustrated in FIG. 2. Both EMT and ICU conditioning units 13, 19 are shown as a single diagrammatic block 13, 19 connected to heat exchanger 17 via tubing 15. Units 13, 19 include a coolant reservoir 21 which contains a supply of liquid coolant 23. A liquid pump 25 pumps liquid coolant 23 from reservoir 21 into umbilical tubing 15 which carries the liquid coolant to conformal heat exchanger 17.

Liquid coolant returns from conformal heat exchanger 17 passing back through umbilical tubing 15 to the EMT/ICU conditioning unit 13, 19. A control valve 27 within units 13, 19 channels the return liquid coolant along a pathway 29 to a cooling heat exchanger 31, and then back into coolant reservoir 21. Cooling heat exchanger 31 interacts with a removable ice cartridge 33 to cool the liquid coolant.

Additionally, control valve 27 is able to channel the return liquid coolant along a pathway 35 which bypasses cooling heat exchanger 31, and returns the liquid coolant to coolant reservoir 21. Thus, the bypassing of cooling heat exchanger 31 avoids cooling of the return coolant.

Control valve 27 is operable to control the temperature of the liquid coolant to within a particular range, as for example, between 5° C. (41° F.) and 25° C. (77° F.). The coolant temperature is controlled by regulating the coolant's circulation, either along pathway 29 through cooling heat exchanger 31 (chilling the coolant) or bypassing it, completely or partially along pathway 35. As used in EMT conditioning unit 13, control valve 27 may vary the percentage of coolant moving along bypass pathway 35 from 0 percent to 100 percent.

The conformal heat exchanger 17 may be pneumatically pressurized by the conditioning units 13, 19 in order to improve surface contact and thermal conductivity with the patient. An air pump 37 within unit 13, 19 provides pressurized air to conformal heat exchanger 17 via tubing 15. In addition, air pump 37 may supply pressurized air to cooling heat exchanger 31.

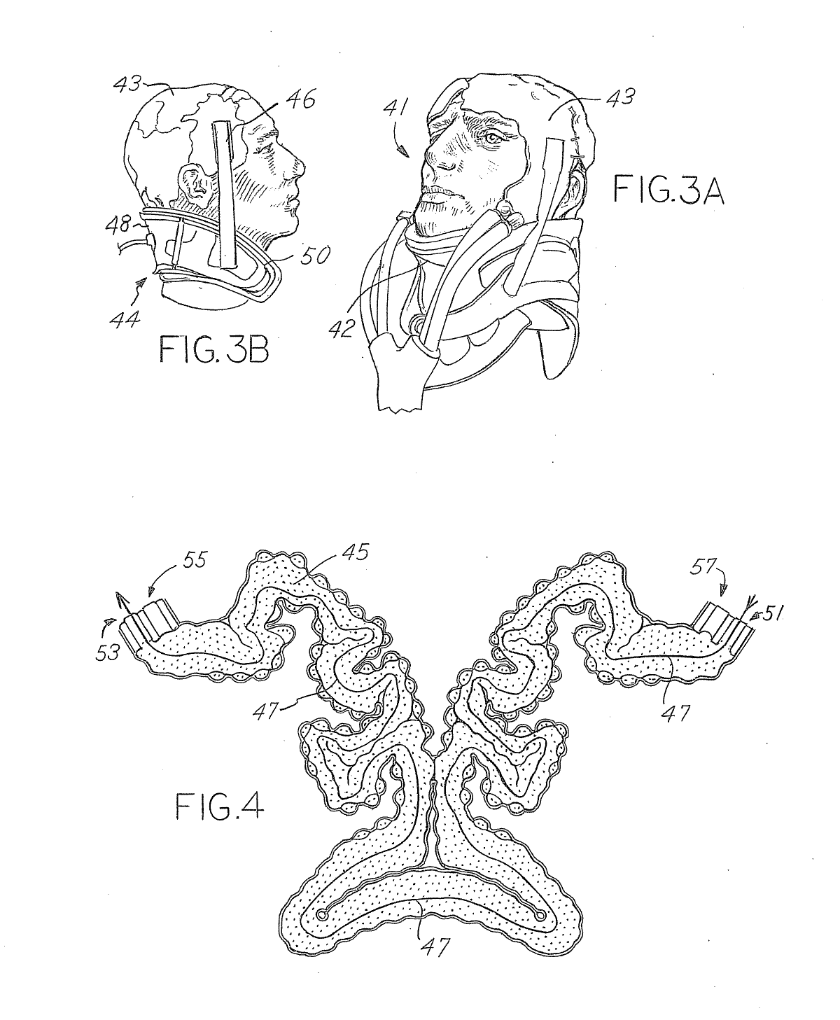

Conformal heat exchanger 17 may be constructed to cover the head of a patient, as represented in FIG. 2. Alternatively, conformal heat exchanger 17 may be constructed and shaped so as to cover the chest of the patient, or another portion of the patient's body. As shown in FIG. 3A, a headliner assembly 41 may be used having conformal heat exchanger 17. Headliner assembly 41 is described in more detail in pending U.S. patent application Ser. No. 13/373,061, filed Nov. 3, 2011, which is incorporated herein by reference. See also U.S. Pat. Nos. 7,509,692 and 7,565,705, which are incorporated herein by reference.

Headliner assembly 41 includes a cap portion 43 and a neck brace 42. Cap portion 43 is a thin, compliant, “one-size-fits-all” conformal heat exchanger, which may be sized to cover the cranium, neck including the carotid triangle, and sides of the face. Cap portion 43 is shaped to fit snugly over the head of the patient. Neck brace 42 may be formed from a modified Aspen Vista Cervical Collar assembly. As shown in FIG. 3B, a dual hinged non-metallic collar 44 may be used, which is compatible with MRI, fMRI imaging. The collar 44 uses compressible elastomeric springs to create a frictional hold between the hinge elements. It may be used for sports side line cooling of an individual.

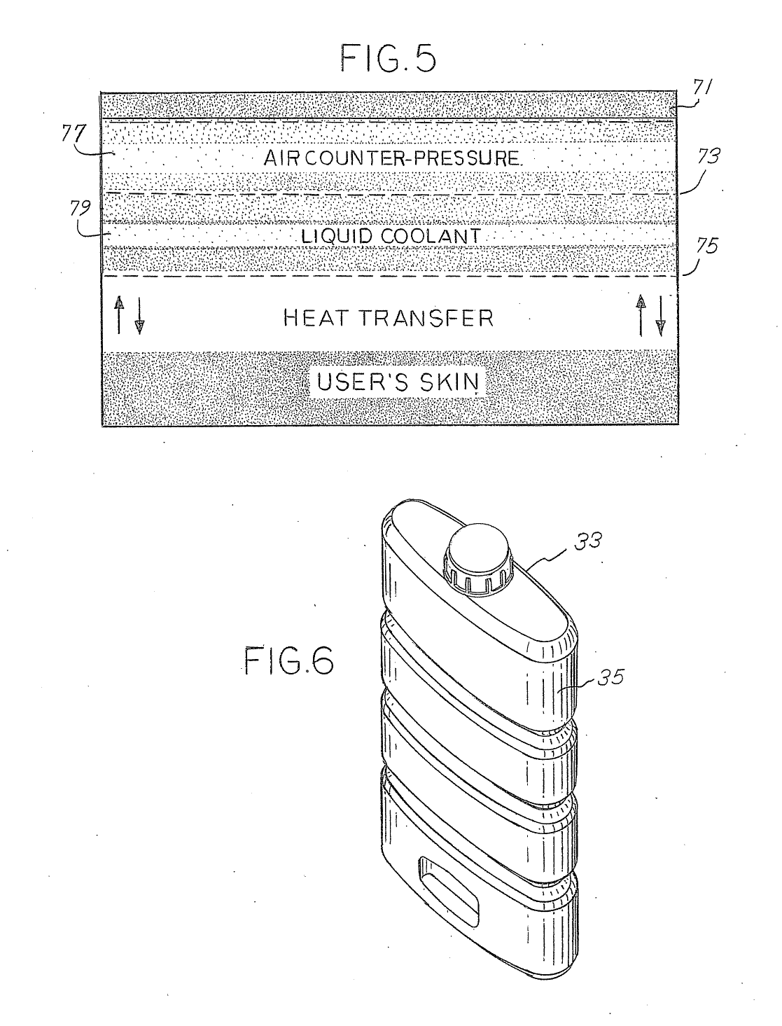

As shown in FIG. 4, the cap portion comprises a serpentine panel 45 which is laced together by an elastic cord (not shown), which gives cap portion 43 the structure and flexibility to fit a wide range of patient head sizes. The exterior surface of the cap portion may be made from a Velcro™-compatible hook/loop material, which facilitates the attachment of cap portion 43 to a modified Aspen Vista Universal Cervical Collar (FDA Class I Medical Device, Regulation Number 890.3490 “Orthosis, Cervical”) with compatible fabric on its inner lining. Alternatively, the headliner may be bonded to the Aspen Vista Collar or other collar devices. Further, straps 46 (FIG. 3B) may have Velcro compatible surface areas to further aid in securing collar 44 to cap portion 43.

A heat transfer liquid pathway (represented by line 47 in the dotted pathway area in FIG. 4) is routed through cap portion 43 and disposed in a heat transfer relationship with the head of the patient. An inlet 51 (FIG. 4) is provided where the heat transfer liquid enters the liquid pathway within cap portion 43. An outlet 53 (FIG. 4) is provided where the heat transfer liquid exits the liquid pathway of cap portion 43. Air ports 55, 57 each provide for air inlet-outlet, as described below, and more specifically, in the above-referenced U.S. patent application Ser. No. 13/373,061. This '061 patent application describes the physical connection between the cap portion and the neck collar.

Referring to FIG. 5, cap portion 43 (FIGS. 3A, 3B) is constructed of three layers of heat-sealed and coated nylon textiles. The material layers are: an outer layer 71, a middle layer 73 and an inner layer 75. The three layers 71, 73, 75 create two inner paths 77, 79. Path 79 carries the liquid coolant to cause heat transfer from the patient. Path 77 carries air providing pneumatic counter-pressure, for causing intimate contact between the heat exchanger (specifically, inner layer 75) and the patient to enhance heat transfer. Outer layer 71 of the cap portion is made from nylon with a soft fabric exterior (nylon loop compatible with a standard hook, e.g. Velcro™ material) and a polyurethane coated nylon fabric interior. Between the soft fabric exterior (the loop material) and the polyurethane coated nylon fabric interior is a thin layer of foam. Both the loop material and foam increase the thermal insulation of the assembly. Middle layer 73 is a dual-sided polyurethane coated nylon. Inner layer 75 is nylon fabric with a single side, the side closer to path 79, being coated with polyurethane.

Referring again to FIG. 2, ice cartridge 33 serves as a heat sink for both the EMT and ICU conditioning units 13, 19. The ice cartridge is constructed as a separate unit so as to be quickly removable from units 13, 19 so that cartridge 33 may be frozen at a location remote from the unit. The ice cartridge when positioned within the units 13, 19 chills the liquid coolant as the coolant circulates through coolant heat exchanger 31. Heat exchanger 31 may be placed adjacent ice cartridge 33 so as to wrap around the outside of ice cartridge 33. Further, heat exchanger 31 may be formed similar to the cap portion 43 to include two paths: (1) an outer path, similar to outer path 77 (described in reference to FIG. 5), for carrying air to provide pneumatic counter-pressure for causing contact with the ice cartridge and (2) an inner path, similar to inner path 79 (FIG. 5), for carrying the liquid coolant.

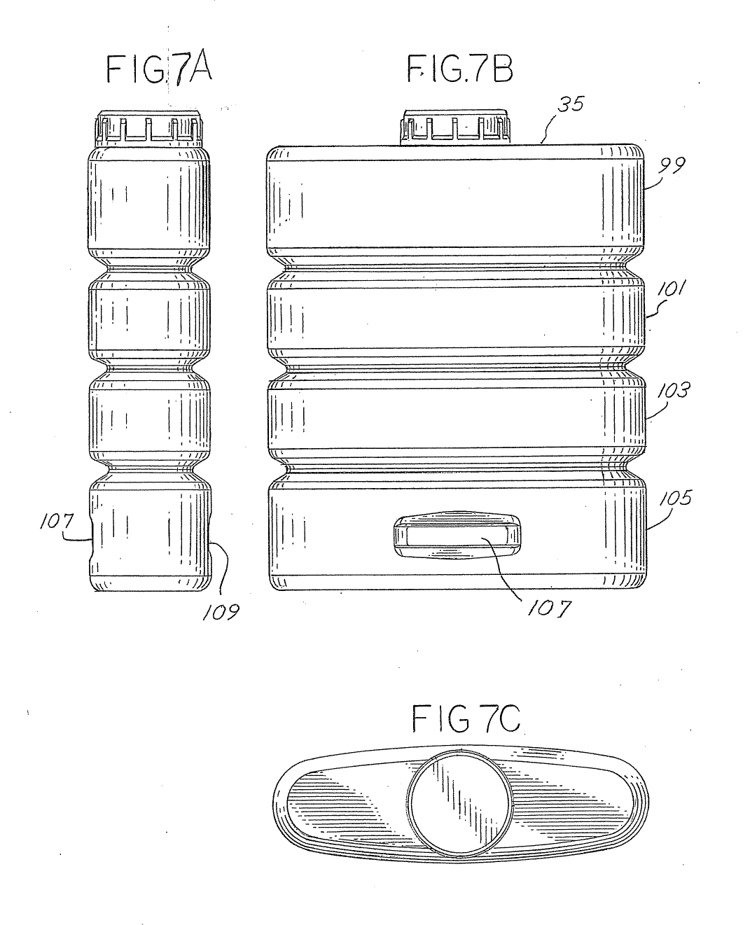

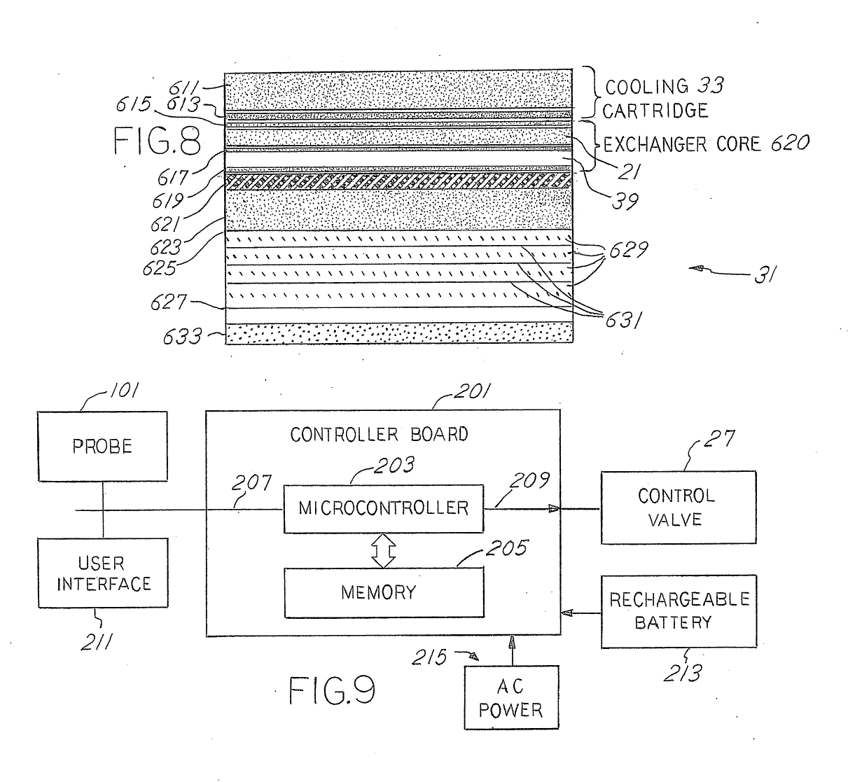

As shown in FIG. 6, and FIGS. 7A-C, ice cartridge 33 is a generally rectangular shaped when viewed from its side while, when viewed from the top or bottom, the cartridge is of ellipsoidal shape. The container is of a size for holding approximately one gallon of fluid. The fluid in cartridge 33 is a refreezeable liquid that may be a mixture of water (88.5%), propylene glycol (11%), tincture of iodine (0.25%), a wetting agent (0.25%) and a trace amount of blue tint. The fluid mixture may be changed to achieve different freeze points depending on the application.

Ice cartridge 33 is constructed from a high density polyethylene. Its configuration, as shown in FIGS. 7A-C makes the cartridge tolerant to multiple freeze thaw cycles. Cartridge housing 35 is shaped with accordion-like expansion rings 99, 101, 103, 105. These expansion rings and the cartridge's ellipsoid shape provide additional flexibility for repeated freezing and thawing. Also, hand indentation grooves 107, 109 may be formed on the lower portion of housing 35 to facilitate handling.

Ice cartridge 33 is removably insertable into the EMT conditioning unit 13, or into the ICU conditioning unit 19, and thereafter is contained within the unit 13, 19. Heat exchanger 31 is wrapped around the cartridge, or disposed such that the surfaces of heat exchanger 31 are in a heat transfer relationship with the ice cartridge. As will suggest itself, other ways may be used to engage the cooling exchanger with the cartridge. Further the pressurized air from air pump 37 (FIG. 2) may be relieved from cooling heat exchanger 31 in order to allow the removal and receiving of the ice cartridge.

Referring to FIG. 8, cooling cartridge 33 contains a fluid (represented as a block) 611. Fluid 611 is initially in a frozen state. Cartridge 33 includes an outer wall 613 against which the cooling heat exchanger 31 is positioned. Cooling heat exchanger 31 includes an exchanger core 620 which is formed from three layers, including an inner layer 615, a middle layer 617 and an outer layer 619. A liquid coolant 21 flows between layers 615, 617, and pressurized air 39 flows between layers 617, 619. The inner layer 615 is formed from a nylon outer layer (adjacent the cartridge wall 613) and an urethane inner layer (for contact with the liquid coolant 21). The middle layer 617 is formed from an urethane outer layer (for contact with the liquid coolant 21), a nylon middle layer and a urethane outer layer (for contact with air 39). The outer layer 619 is formed from a nylon layer having a urethane inner layer (for contact with air 39).

The heat exchanger outer layer 619 is secured to a thermoplastic wall 621. A 3M Thinsulate layer 623 is secured to wall 621 and to a composite layer formed of two nylon fabric layers 625, 627. Between the two nylon fabric layers 625, 627 are four layers of scrim 629, and three layers of aluminized Mylar 631. These alternating layers of aluminized Mylar and scrim form a heat radiation ebarrier similar to that used in space suit micrometeoroid garments.

The EMT unit 13 additionally includes an outer layer of a heat formable fabric foam composite 633 which serves to protect the inner layers. The ICU unit 19, on the other hand, includes a thermoplastic wall (not shown) in place of layer 633.

Referring again to FIG. 1, a patient temperature probe jack 101 is positioned to extend out from the front of the housing of the ICU conditioning unit 13. Temperature probe jack 101 is connectable to the patient in order to obtain patient temperature information and provide the temperature information to unit 19. Temperature probe jack 101 is positioned so that it may obtain a recording of the patient's core temperature. The patient's changing temperature may be used to control the rate of cooling performed by conditioning unit 19.

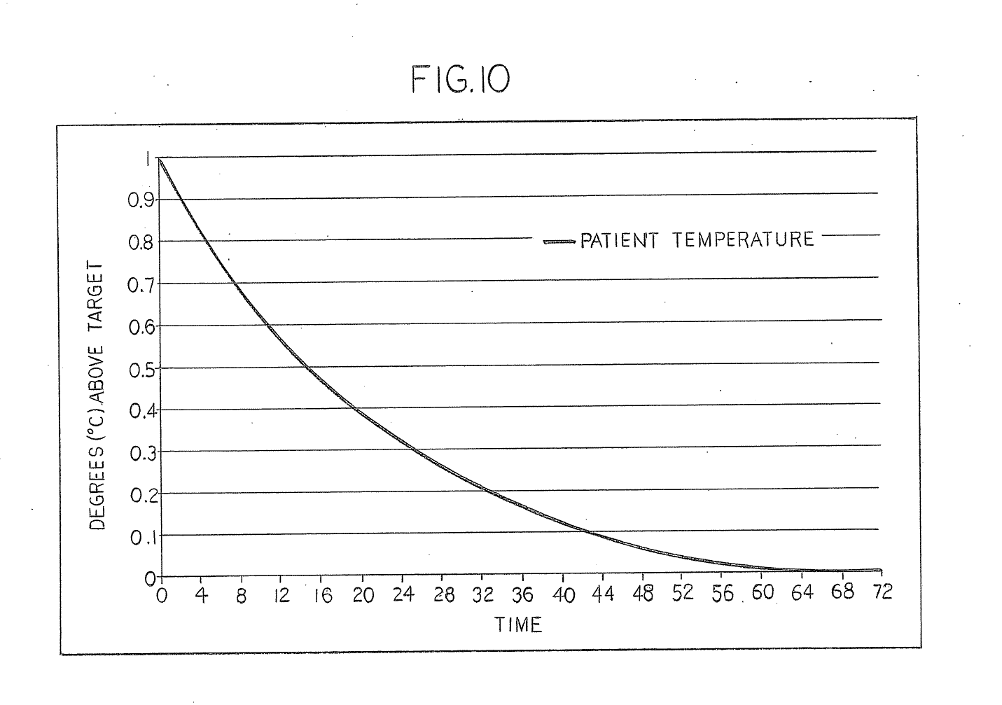

Referring to FIG. 9, the ICU conditioning unit includes a controller board 201, which in the illustrated example includes a microcontroller 203. Microcontroller 203 may be a microprocessor, programmable logic device or other computational device, or a general purpose computer. Instructions and data to control operation of microcontroller 203 are stored in a memory 205 which is in data communication with microcontroller 203. Memory 205 may include both volatile and non-volatile memory. Temperature probe 101 communicates with microcontroller 203 along a conductor input 207. Microcontroller 203 outputs control instructions and other data along a conductor 209.

Microcontroller 203 begins to control the patient's temperature once the patient's core temperature is one degree centigrade above a set point. The core temperature may reach less than one degree Celsius above the set point depending on the amount of time that the patient was attached to the EMT unit before being transferred to the ICU unit. The time required for the EMT unit to drop the patient's temperature depends on the weight of the patient. During the time that the patient's core temperature is above the set point by more than one degree centigrade, the microcontroller is in a FULL COLD mode and does not control the rate of core temperature drop. When the patient's temperature reaches a point that is one degree Celsius above a set point temperature for the patient, the microcontroller begins to adjust the coolant temperature using the control valve 27. Until that time at which the patient's temperature is one degree above the set point, ICU conditioning unit 19 remains in a FULL COLD mode with the control valve 27 providing the coolant along the path through the cooling heat exchanger 31. This coolant adjustment over a single degree is performed in accordance with a number of temperature set points, for example, 72 set points, shown below in Table 1. As understood, the decimal numeral in the right column of Table 1 represents a value used by the microcontroller 203 to regulate the control valve 27.

TABLE 1

| 5 min increments | Deg C. above setpoint | Micro Controller Table |
|---|---|---|
| 0 | 0.995988 | 996 |
| 1 | 0.948308 | 948 |
| 2 | 0.903152 | 903 |
| 3 | 0.860373 | 860 |
| 4 | 0.819832 | 820 |
| 5 | 0.781396 | 781 |
| 6 | 0.74494 | 745 |
| 7 | 0.710343 | 710 |
| 8 | 0.67749 | 677 |
| 9 | 0.646275 | 646 |
| 10 | 0.616594 | 617 |
| 11 | 0.58835 | 588 |
| 12 | 0.561452 | 561 |
| 13 | 0.535814 | 536 |
| 14 | 0.511354 | 511 |
| 15 | 0.487996 | 488 |
| 16 | 0.465668 | 466 |
| 17 | 0.444305 | 444 |
| 18 | 0.423843 | 424 |
| 19 | 0.404224 | 404 |
| 20 | 0.385396 | 385 |
| 21 | 0.367307 | 367 |
| 22 | 0.349913 | 350 |
| 23 | 0.33317 | 333 |
| 24 | 0.317041 | 317 |
| 25 | 0.301491 | 301 |
| 26 | 0.286486 | 286 |
| 27 | 0.271999 | 272 |
| 28 | 0.258003 | 258 |
| 29 | 0.244476 | 244 |
| 30 | 0.231397 | 231 |
| 31 | 0.218749 | 219 |
| 32 | 0.206514 | 207 |
| 33 | 0.194681 | 195 |
| 34 | 0.183238 | 183 |
| 35 | 0.172175 | 172 |
| 36 | 0.161485 | 161 |
| 37 | 0.151161 | 151 |
| 38 | 0.141198 | 141 |
| 39 | 0.131593 | 132 |
| 40 | 0.122344 | 122 |
| 41 | 0.11345 | 113 |
| 42 | 0.104908 | 105 |
| 43 | 0.0967206 | 97 |
| 44 | 0.0888868 | 89 |
| 45 | 0.0814079 | 81 |
| 46 | 0.0742848 | 74 |
| 47 | 0.0675187 | 68 |
| 48 | 0.0611105 | 61 |
| 49 | 0.0550608 | 55 |
| 50 | 0.04937 | 49 |
| 51 | 0.0440376 | 44 |
| 52 | 0.0390627 | 39 |
| 53 | 0.0344432 | 34 |
| 54 | 0.0301763 | 30 |
| 55 | 0.0262576 | 26 |
| 56 | 0.0226818 | 23 |
| 57 | 0.0194417 | 19 |
| 58 | 0.0165288 | 17 |
| 59 | 0.0139324 | 14 |
| 60 | 0.0116402 | 12 |
| 61 | 0.00963767 | 10 |
| 62 | 0.0079079 | 8 |
| 63 | 0.00643167 | 6 |
| 64 | 0.00518717 | 5 |
| 65 | 0.00414985 | 4 |
| 66 | 0.00329227 | 3 |
| 67 | 0.00258398 | 3 |
| 68 | 0.00199131 | 2 |
| 69 | 0.00147728 | 1 |
| 70 | 0.00100139 | 1 |
| 71 | 0.000519499 | 1 |
| 72 | 0 | 0 |

These set points of Table 1 represent a “feedback curve” which illustrates how the patient's temperature is to be decreased by 1 degree over time. Microcontroller 203 uses each of the 72 temperature set points which are stored in memory 205. One set point is used every five (5) minutes to decrease the patient's temperature over that five minute period. Microcontroller 203 adjusts the coolant temperature downwardly by operating control valve 27 while monitoring the patient's temperature. Microcontroller 203 monitors the patient's temperature with respect to the five minute set point temperature. The patient's temperature is thus decreased each five minutes, starting at a time when the patient's temperature is 1 degree over a set point temperature. Other temperatures and sampling time intervals may be used, and other than a 1 degree decrease may be used, as will suggest itself.

As will suggest itself, the microcontroller 203 may be used to rewarm a patient to slowly return a patient's core temperature to normal.

Control valve 27 in the EMT unit may operate differently than the control valve 27 in the ICU unit. Control valve 27 may be a digitally controlled solenoid operated pinch valve used in ICU conditioning unit 19, and, which receives control signals from output 209 of microcontroller 203. The control valve of the ICU unit may have two positions: OPEN and CLOSED, as described below.

Control valve 27 as used in the EMT conditioning unit 13, may be manually controlled by the user. The control valve 27 which is used in the EMT unit may be comprised of a cam (not shown) that is manually moveable relative to two flexible tubes (not shown). Manual movement (e.g., rotation) of the cam linearly squeezes the two flexible tubes to a degree dependent on the position of the cam. With the cam in a first position, one of the flexible tubes is fully opened and the other flexible tube is fully closed. With the cam in a second position, the one flexible tube is opened approximately two-thirds (⅔) and the other flexible tube is opened approximately one-third (⅓). With the cam in the third position, the one flexible tube is opened approximately one-third (⅓) and the other flexible tube is opened approximately two-thirds (⅔). This provides for a COLDEST, COLDER and COLD settings of the EMT, as describe below.

Additionally, the cam of control valve 27 may be adjusted to more than three positions, and may be linearly adjusted between two points. Valve 27 may have a temperature range of 180° that would allow a full range from FULL COLD to FULL WARM. As another example, the EMT may be constructed to limit the temperature range to 135°, so that temperature range is limited linearly to moderately cold through full cold. As will suggest itself, two cams may be used, one cam for one flexible tube and the other cam for the other flexible tube. Other manually controlled pinch valves may be used as well.

Referring to FIG. 2, the solenoid operated pinch valve 27 of the ICU unit, when in a CLOSED position allows flow of liquid coolant directly into the cooling heat exchanger 31 (pathway 29 is open and pathway 35 is closed). When pinch valve 27 is in an OPEN position, the returning (warmed) coolant is directly returned to reservoir 21 (pathway 29 is closed and pathway 35 is open). The reservoir coolant temperature is the result of the combination of the two coolant temperatures of coolant moving along the two pathways 29, 35. The resulting (outlet) temperature of the coolant modulates the cooling rate of the patient. Reservoir 21 is of sufficient size to allow both cold and warm liquid to mix before returning to liquid pump 25.

Referring to FIG. 9, the cooling process is performed by microcontroller 203 in the ICU unit, with input at 207 from the indwelling patient probe 101 (indicating the patient's core temperature). Other parameters (including those of table 1 above) may be stored in memory 205, including user input parameters entered via a touchscreen user interface 211. Such parameters include treatment parameters, as for example, a target patient temperature and a time duration.

The ICU unit delivers coolant at the coldest (FULL COLD) setting until patient core temperature has been reduced to a level at 1° C. above the target patient temperature. Below this point, within 1° C. above the target patient temperature, the system initiates an automatic temperature control algorithm, which adjusts coolant temperature by shuttling the solenoid valve 27 between FULL COLD/valve closed, and FULL WARM/valve open in order to approach the target patient temperature in a roughly asymptotic-type curve. To modulate coolant temperature and approach target temperature asymptotically, the control algorithm cools or warms the liquid coolant by modulating its flow. Specifically, ICU microcontroller 203 commands the solenoid valve to open or close, modulating the flow of liquid coolant through two liquid paths: first, through the cooling heat exchanger to cool down the liquid in circulation and to provide increased cooling therapy for the patient (FULL COLD/valve closed), or in a mode that bypasses the heat exchanger and only circulates liquid without lowering its temperature (FULL WARM/valve open). The precise mixture of FULL COLD to FULL WARM (valve closed to valve opened) is determined by the microcontroller algorithm, based on the patient's core temperature relative to the asymptotic curve. If, for example, the patient temperature is trending above the curve (i.e. not cooling fast enough), the system will automatically increase the valve ratio of FULL COLD to FULL WARM in order to increase cooling and return the patient temperature to the curve.

Microcontroller 203 uses the values shown above in Table 1 of seventy-two 5-minute increments (0-72), each with an associated temperature set point (effectively defining a temperature curve from 1 degree to 0 degrees over a 360 minute period). The frequency of the valve action may be 20 seconds to assure proper mixing of the cooled and warm liquid to have the resultant temperature of the liquid that exits being at an average temperature. This curve is asymptotic in that the curve approaches zero, the base horizontal of 0 degrees, as shown in FIG. 10. This regression curve of FIG. 10 sets the desired temperature. As will suggest itself, other curves may be used. The formula that sets the regression curve may be changed depending on the patient. The microcontroller begins by looking at the first set point temperature for the 5 minute period in the table (e.g., 0.995988) and then looks at the patient's temperature (e.g., 1 degree) and then adjusts the control valve to approach the desired patient temperature. The solenoid valve has only two positions (1) FULL COLD (solenoid valve closed) and (2) FULL WARM (solenoid valve open). The solenoid valve's normal position is closed. The dwell time of the control valve is adjusted in one of the two positions ON/OFF. Initially, the time increment may be 120 seconds. Within the time increment, the percentage of FULL WARM (solenoid open) to FULL COLD (solenoid closed) is varied as a function of the deviation of the patient's temperature from the control curve (set temperature values). After 5 minutes, the temperature set point (e.g., 0.995988 shown in Table 1) changes (e.g., to 0.948308 shown in Table 1), resulting in an adjustment of the valve.

The solenoid valve cyclic frequency is related to the volume of the coolant reservoir so that substantial mixing occurs between the FULL COLD and FULL WARM coolant returning to the reservoir. The resultant coolant is then returned to the headliner assembly.

Referring to FIG. 11, the EMT conditioning unit may include a control-display panel 901. An ON-OFF power button 903 controls power to the EMT conditioning unit 13. In addition, a MUTE button 905 separately turns on/off an audio beeper (not shown) which is used to provide audio alerts, for example, when the cartridge temperature is too high. Small green indicator lights 907, 909 are illuminated when the respective controls 903, 905 are actuated to the ON position. Audio visual display 911 is for “change cartridge” and audio visual display 913 is for “change battery.” Circuitry (not shown) monitors battery level and exit temperature of the coolant. Cartridge change-out is based on the temperature of the liquid leaving the heat exchanger. Change-out warnings may occur at 60° F., for example. When either the “changecartridge” or “changebattery” displays 911, 913 is illuminated (and flashing), an audible beeping is engaged (unless the Mute button has been actuated). The indicator light 909 will not illuminate in this case.

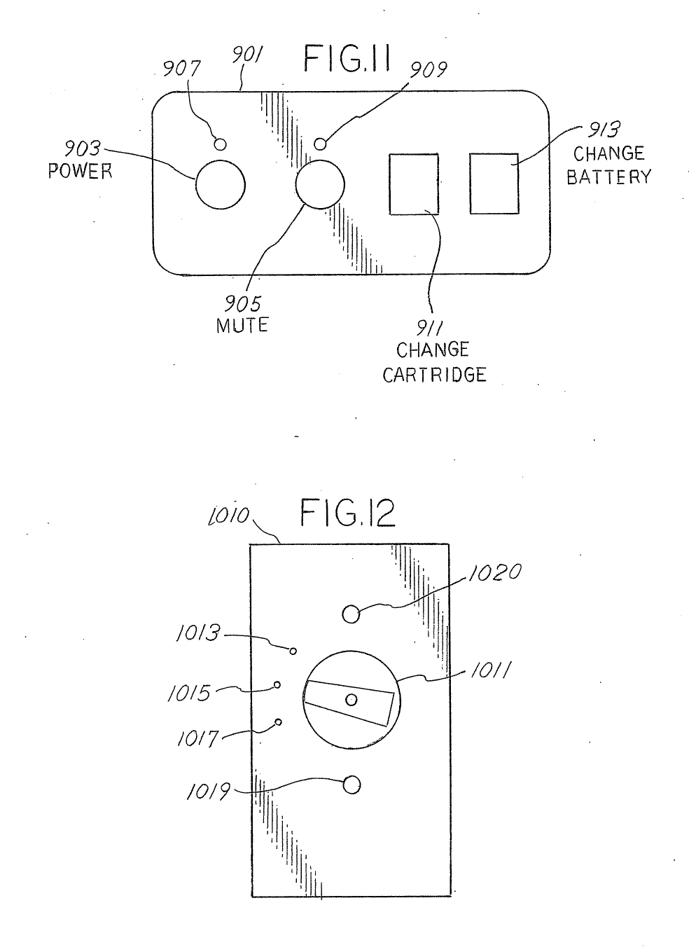

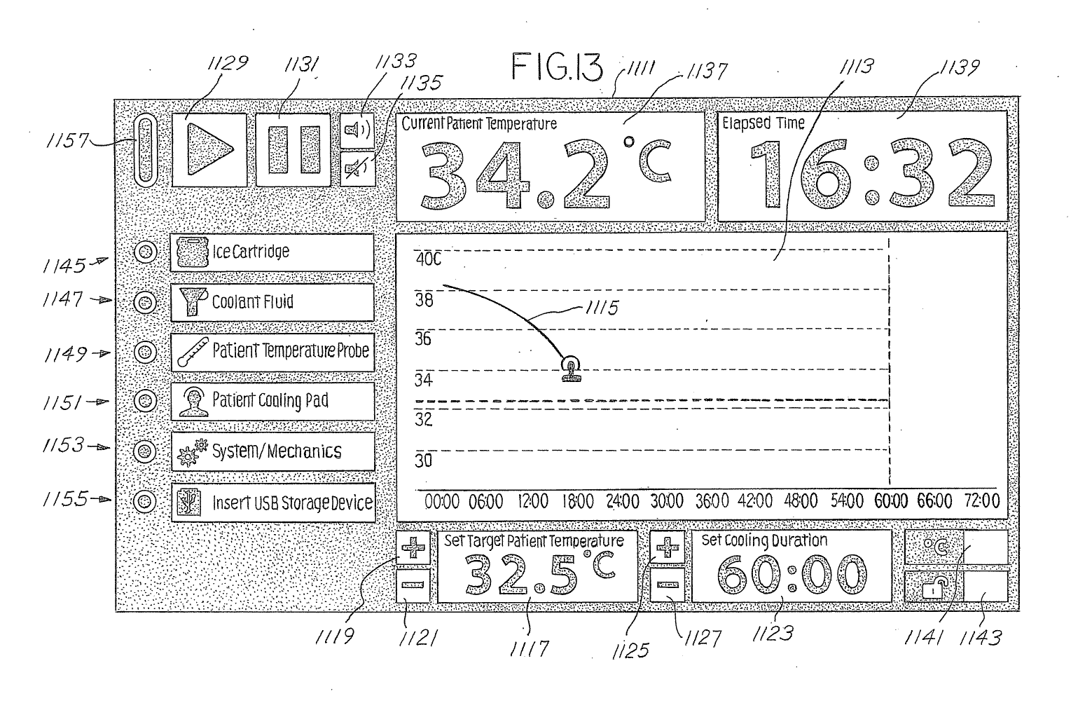

Referring to FIG. 12, the EMT conditioning unit may include a control-display panel 1010 having a control knob 1011. Control knob 1011 is rotatable by the user to select an analog range of settings corresponding to a temperature range of 180 degrees, for example. A range of dot indicators on the panel provide for (COLD) 1013, (COLDER) 1015 and (COLDEST) 1017, and thus provide a visual indication of temperature setting. Alternatively, a varying thickness line may be used in place of indicators 1013, 1015 and 1017. Manual linear movement of knob 1011 controls the position of control valve 27 so as to move its cam to the first, second or third position as described above (or to positions in between to provide a linear range) so as to control the percentage of warm (return) liquid that flows directly to the reservoir without flowing through the cooling cartridge heat exchanger. The range of the control knob 1011 may be limited to less than 180 degrees of temperature depending on the use of the EMT unit. Most medical applications have a range from −45 degrees to −170 degrees. Further, the final 10 degrees of range may require the depression of a button 1020.

In addition, control knob 1011 may be used to select a setting 1019 in order to activate a refill procedure (“Auto-Fill”) in which the polarity of coolant pump 25 is reversed and pump 25 is activated in order to refill reservoir 21 (as discussed below). A button 1020 must be depressed in order to rotate control knob 1011 to the Auto-Fill setting 1019.

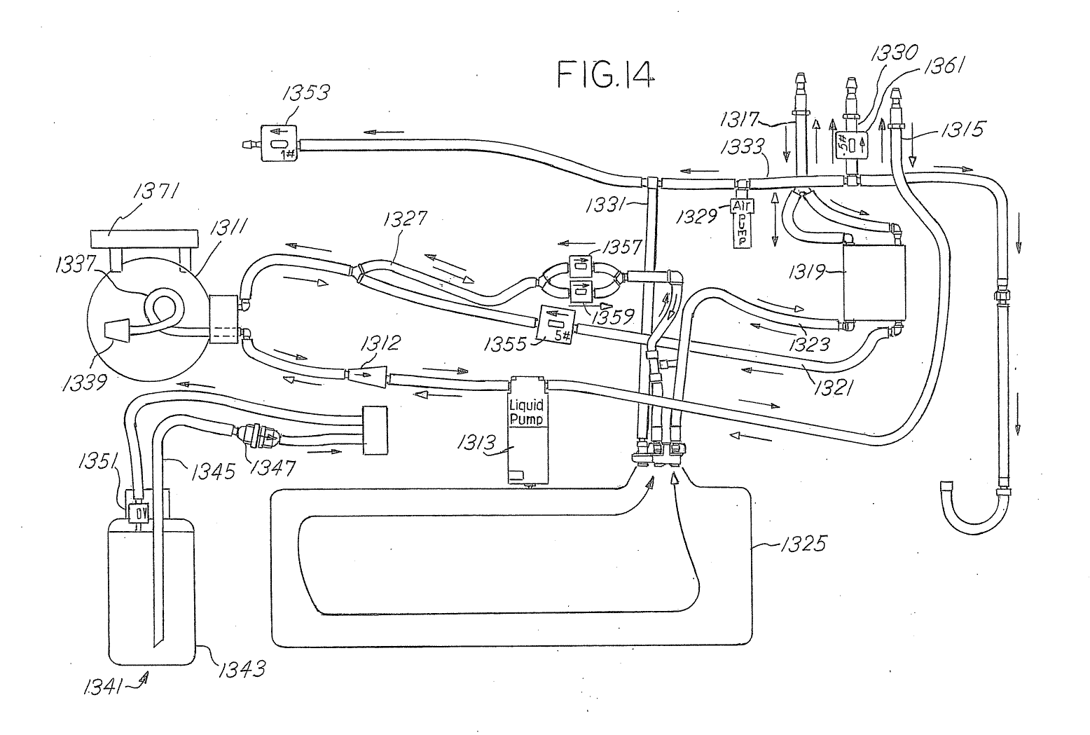

Referring to FIG. 13, ICU conditioning unit 19 may include a user control panel/display formed of a touch screen monitor 1111. A temperature graph 1113 may be displayed on monitor 1111, as shown in FIG. 13. Temperature graph 1113 displays a curve 1115 showing user temperature versus elapsed time. The user temperature is the temperature monitored from the patient via probe 101 (FIG. 1). A continuing curve portion (not shown in FIG. 12) may be added to curve 1115 so as to indicate a predicted curve of future temperatures through a set time. Microcontroller 203 of the ICU conditioning unit generates the graph from monitored temperature values and may provide the continuing curve portion to complete the graph along a general line of descent toward the target temperature.

Touch screen monitor 1111 includes a target temperature area 1117. A default temperature value as the treatment setting may be loaded into memory 205 (FIG. 9) and then displayed in area 1117, as for example, a target user temperature of 35° C. (95° F.). To modify the target user temperature, two touch buttons 1119, 1121 are provided to the left of target temperature area 1117. To increase the target temperature, button 1119 is used, and to decrease the target temperature, button 1121 is used. The target temperature is changed in 0.5° C. (1.0° F.) increments, for example, between 30° C. (86° F.) and 37° C. (98.6° F.).

Touch screen monitor 1111 also includes a cooling duration area 1123. A default duration value may be loaded into memory 205 (FIG. 9) and then displayed in area 1123, for example, a duration value of 60 hours may be set. To modify the cooling duration value, two touch buttons 1125, 1127 are provided to the left of the duration area 1123 to increase (button 1125) or decrease (button 1127) the duration in 6-hour increments between 0 and 72 hours.

Also, touch screen monitor 1111 includes a start button 1129 (having an arrow icon) or a pause button 1131 (having vertical rectangles). When the unit is running, the start button is displayed green and the pause button is displayed grey; when paused, the start button is grey, and the pause button blinks blue-grey.

To increase or decrease the volume of system alarms and warnings (which may be generated from an audio speaker (not shown in FIG. 13)), users may press a HIGH-LOW VOLUME button 1133. The speaker icon on button 1133 will visually change to reflect whether the speaker volume is set to a HIGH or a LOW by displaying a number of partial circles on button 1133. Alternatively, users may mute system alarms by pressing a MUTE button 1135. The icon on the MUTE button will glow blue when muted.

The user's current temperature, based on the reading from the indwelling temperature probe 101, is displayed in area 1137 at the top center of the touch screen monitor.

Elapsed time is displayed in area 1139 in hours and minutes (HH:MM) in the top right corner of the touch screen monitor, and represents the total duration of active cooling in the current treatment. When the system is paused via button 1131, the elapsed time counter (not shown) is paused as well and display 1139 remains fixed to the time duration at the point of the pause.

To switch the displayed units between degrees Celsius and Fahrenheit (° F.), a button 1141 may be used. Also, the user may lock cooling treatment parameters during operation by toggling a button 1143. When locked, touch screen controls will not respond to contact, but the user temperature, elapsed time, and status displays will continue to function normally. ICU conditioning unit 19 continually monitors its performance and the health of major components, and issues warnings or alarms to notify the user of conditions that may interfere with user safety or system performance. A warning may notify a user of an error condition that can be corrected or cleared by the user, and does not pose an immediate hazard to the user, or device. An alarm may notify the user of an error condition that cannot be corrected by the user.

Visual warnings and alarms may be provided by status indicators positioned on the left side of touch screen monitor 1111, which illuminate and display relevant information when an issue is detected. There are six primary indicator areas shown in FIG. 12: Ice Cartridge 1145, Coolant Fluid 1147, User Probe 1149, Conformal Heat Exchanger 1151, System/Mechanics 1153 and USB Interface 1155. Each indicator may comprise two parts: a descriptive box with Warning—or Alarm-specific (e.g., “Replace Ice Cartridge” or “Replace User Probe”) information and colored background (grey for normal, amber for Warning, or red for Alarm) and a corresponding circular indicator light (green for normal, amber for Warning, or red for Alarm). In addition to the condition-specific Status Indicators, a single Master Status Indicator 1157, located at the top left corner of the User Therapy Screen may display a top-level summary of system status.

Referring to FIG. 14, EMT conditioning unit 13 includes a coolant reservoir 1311 from which a liquid pump 1313 extracts coolant and pumps the coolant out of an exit port 1315. Arrows shown in FIG. 14 having solid arrow heads to indicate flow during normal operation. Arrows having an open arrowhead indicate flow during the refill mode. A coolant output port 1315 is attachable to the umbilical tubing 15 (FIG. 2). The coolant is returned to EMT conditioning unit 13 via an input port 1317 which is attachable to tubing 15 (FIG. 2). The returned coolant enters a control valve 1319 and follows fully or partially a warm path along tube 1321 or a cold path along tube 1323. Control valve 1319 may be manually operated (e.g., by knob 1011, FIG. 11) to guide the coolant flow in tubes 1321 and 1323. Tube 1321 is connected to reservoir 1311, while tube 1323 is connected to a cooling heat exchanger 1325 which is wrapped about an ice cartridge (not shown). From cooling heat exchanger 1325, the coolant passes along tube 1327 to reservoir 1311.

An air pump 1329 may provide compressed air along tube 1331 to the cooling heat exchanger 1325, and provide compressed air along tube 1333 to an exit port 1330 which is connected to tubing 15 leading to conformal heat exchanger 17 (FIG. 2). Additional air passageways may be provided as will suggest itself.

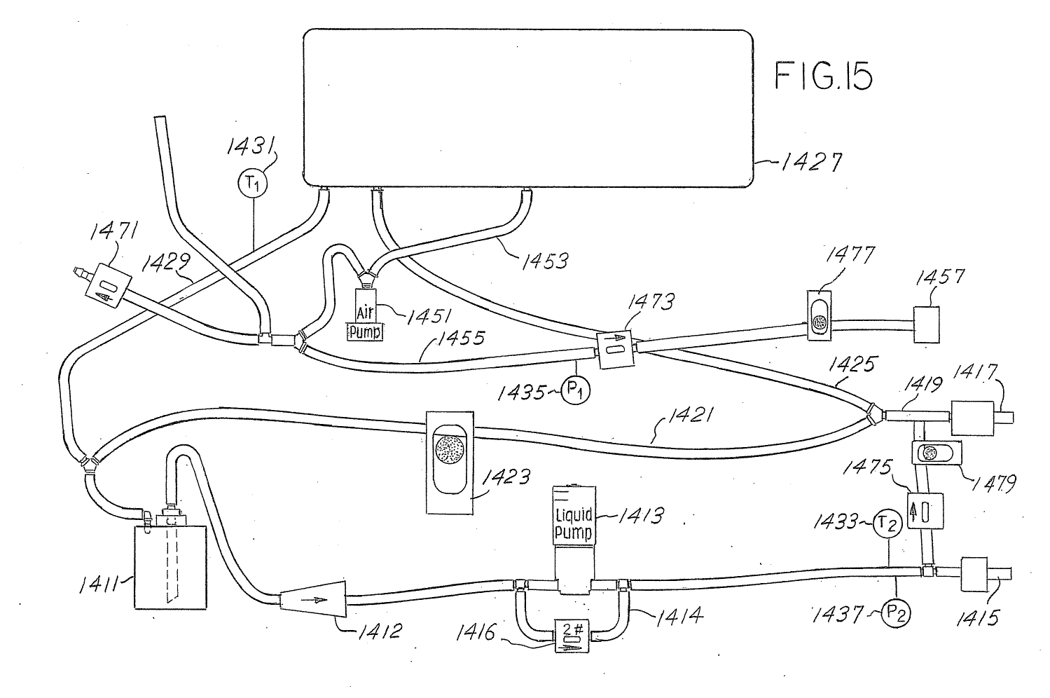

Referring to FIG. 15, ICU conditioning unit 19 includes a coolant reservoir 1411 from which a liquid pump 1413 extracts coolant and pumps the coolant out of an exit port 1415 which is attachable to tubing 15 (FIG. 2). An inline filter 1412 is disposed between reservoir 1411 and liquid pump 1413. In addition, a bypass tubing 1414 and check valve 1416 keeps reservoir 1411 from being overpressurized above two (2) psig during an auto-fill process. The coolant is returned to the ICU conditioning unit 13 at input port 1417 which is attachable to tubing 15 (FIG. 2). The returned coolant enters a tube 1419 and will flow along tube 1421 back to reservoir 1411 via a control valve 1423. As described above, control valve 1423 may have two positions. If control valve 1423 is closed, then the returned coolant will flow along tube 1425 to the cooling heat exchanger 1427 and then through tube 1429 to reservoir 1411.

An air pump 1451 may provide compressed air along tube 1453 to cooling heat exchanger 1427, and provide compressed air along tube 1455 to an exit port 1457 connected to tubing 15 (FIG. 2) leading to conformal heat exchanger 17 (FIG. 2). Air pump 1451 may be activated through a timer to cycle the ON/OFF times of the pump. For example, the air pump may be turned ON for two minutes and then turned OFF for eight minutes.

As shown in FIG. 15, two temperature sensors 1431, 1433 are located in the coolant pathways so as to measure the coolant temperature in the ICU conditioning unit 19. Likewise, one or more temperature sensors may be located in the EMT conditioning unit 13. One way check valves 1471, 1473, and 1475 may be used to maintain directional flow in one direction, and may maintain a maximum pressure. As will suggest itself, other control valves 1477, 1479 may be provided to control air or liquid flow in the unit.

Referring again to FIG. 14, the liquid exit line from the reservoir 1311 is configured with a spiral tubing 1337 and includes a weighted pick-up 1339 at the tubing's termination end. This permits movement of the tubing within reservoir 1311 and allows gravity to place weighted pick-up 1339 at the bottom of the reservoir regardless of the orientation of the EMT unit. This is aided by use of a spherical shaped reservoir 1311. This permits the EMT conditioning unit to operate in any orientation including upside down.

Air pump 1329 (FIG. 14) and air pump 1451 (FIG. 15) are DC-operated rotary diaphragm air pumps. As will suggest itself, the air pumps may be of different designs. As shown in FIG. 15 for the ICU unit, air pressure sensors 1435 may be placed along the air pathway and used to monitor air pump performance Likewise, fluid pressure sensor 1437 may be placed along the coolant pathway to monitor liquid pump performance. Information from sensors 1435, 1437 may be monitored by microcontroller 203. For the EMT unit, an external test unit may be used to check air pump performance, for example, along an additional air passageway. ICU unit may likewise receive an external test unit.

Referring to FIG. 14, a refill kit 1341 may be used with the EMT unit (or with the ICU unit). A refill bottle 1343 contains coolant and is connectable to the umbilical connection ports 1315 (after the umbilical tubing is disconnected). The polarity on the pump motor 1313 is reversed. Liquid flows from refill bottle 1343 along tube 1345 and into the reservoir 1311. A filter 1347 may be located in tube 1345. The coolant flows in through the outlet port 1315 (now an inlet port) and into the reservoir 1311 forcing air out of the inlet port 1317. The arrows having an open arrowhead show the flow during use of the refill kit 1341. One way check valves 1351, 1353, 1355, 1357, 1359, 1361 may be used to maintain directional flow in one direction. As an example, check valve 1353 (and check valve 1471, FIG. 15) may be used to output air flow at a certain pressure, in order to control the maximum air pressure within the unit.

The cap on the refill bottle is replaced with a cap having valved connectors for attachment to the umbilical ports. The control knob 1011 (FIG. 12) serves as a manually operable switch to reverse the polarity of the pump motor and to drive the motor until the refill bottle is empty, or until visually determined from an externally visible sight glass 1371 (FIG. 14) that shows the level of the coolant. As will suggest itself, a coolant level sensor may be used in reservoir 1311, 1411. The process prevents over filling and spillage, as well as the need for direct access to the reservoir.

Thus, for both the ICU unit and the EMT unit, the internal filters may be backflushed into the refill kit and be captured in the refill kit filter. The fill kit filter element may be replaced as needed. If necessary, the internal filter in the ICU or EMT can be replaced during annual inspections or sooner if required. One of the coolant filters in the ICU may be removed and replaced with a new filter which is provided in the refill kit.

Referring again to FIG. 1, umbilical tubing 15 has an end connector 81 which connects to a corresponding connector 83 of the EMT conditioning unit 13 and connects to a corresponding connector 85 of the ICU conditioning unit 19. Tubing 15 also has a Y-shaped end housing 87 that splits the inlet and outlet liquid pathways and divides the air pathway. Liquid enters along a pathway 89 to heat exchanger 17, and liquid exits along a pathway 91 from the heat exchanger. Air in and air out are provided on both pathways 89, 91. Further, connectors 93, 95 may be provided so as to attach or remove tubing 15 from heat exchanger 17. The tubing 15 may be Tygothane® Precision Polyurethane tubing (Formulation C-210-A which is manufactured by Saint Gobain).

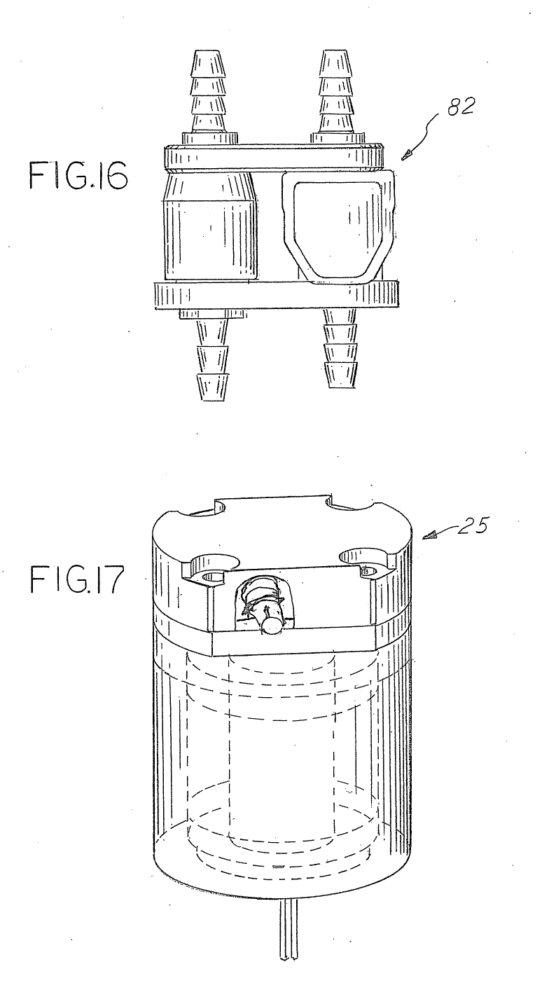

The connectors 81, 83, 85, may be quick-disconnect assemblies that are quickly pushed-on or pulled-off to make or break the connection. The connectors may be either three-port or two port connectors. An example two port connector 82 is shown in FIG. 16.

Connectors 81, 83, 85 may take on various configurations including being formed by injection molding. A plastic cap or dust cover may be used to cover the connectors 81, 83, 85, and such dust cover may be spring loaded.

The connector ports are formed of a number of portions which provide a connection area for receipt of larger but like tube portions from connector assembly 81 which is secured to the umbilical tubing. The fit connection is by frictional fit. A latching mechanism may be used in which a spring loaded male piece with chamfers on both sides of the element locks into a groove on the male portion. The chamfers are angled such that a calibrated pull force is required for the chamfer to ride up the groove and release the male element. The pull force to disconnect may be eight (8) pounds.

Alternatively, a two port connector configuration may be used in conjunction with connection of the umbilical tubing 15 to a body conformal heat exchanger 17. Liquid coolant enters on the right side of exchanger 17 and exits on the left side. Liquid coolant enters and exits on the lower of the two ports. Air enters and exits on both sides of the headliner using the upper one of the two ports. Only the liquid sides (lower) have mechanical push-on pull-off connections. Alternatively, a quick release latch may be used.

Internal control of flow direction and pressures may be accomplished through the use of calibrated-directional check valves. Variable internal pressures are controlled to various elements of the system. For instance, the internal air pressure at the cooling heat exchanger 31 (FIG. 2) may be 1 psig while the pressure at the headliner (heat exchanger 17) may be 0.5 psig.

Referring again to FIG. 9, a rechargeable battery 213 may be used in both the ICU conditioning unit 13 and the EMT conditioning unit 19. The rechargeable battery 213 of EMT unit 13 may be of a different design than the rechargeable battery 213 of the ICU unit 19. Rechargeable battery 213 may be, for example, either a LiO battery or NiMh battery. External AC power, shown at 215, may be connected to board 201 of the ICU unit 13. Further a charger (not shown) may form part of board 201 and be connectable to an external power source 215 in order to recharge battery 213 for either of units 13, 19. Alternatively, alkaline batteries may be used in the EMT unit, or alternatively rechargeable batteries may be recharged externally to the EMT unit.

Memory 205 may store other important information, such as the date of use, the time of day of the use, the duration of the use, the important temperatures of the coolant and the temperatures of the patient. A memory circuit (not shown) may be incorporated into the EMT unit as well.

A number of sensors may be used to monitor the reservoir level, coolant flow rate, air pressure, and coolant temperature. Such monitored values may be stored in memory 205 for use by microcontroller 203 in order to provide display information and visual or audible alarms to the user. In addition, a USB port (not shown) and associated interface circuitry (not shown) may be used for connection of controller board 201 to a remote server (not shown). Microcontroller 203 may then communicate with the remote server or other device via the USB port. Additionally, a personal computer may be connected to the unit via the USB port.

Referring to FIG. 17, liquid pump 25 is a positive displacement, magnetically coupled DC pump, and includes a brushless DC motor. The pump is available from BR Designs of Geneva, Ill. The pump is combined with a magnetic drive and brushless DC motor. The pump rotor is directly attached to the driving gear in the pump housing. In this configuration, the rotor and stator of the motor, in addition to driving the pump, also performs the function of a magnetic clutch. The coolant immerses the gap between the rotor and the stator. This eliminates the need for any dynamic seals or the imposition of a separate magnetic clutch.

Liquid pump 25 makes use of three phase motor technology, and may be controlled by a microprocessor. The speed of the pump may be adjustable as well as reversible. Upon fault detection, the pump may be automatically shut down.

Liquid pump 25 consists of two nearly identical intermeshing gears. One of gears is centered in the pump assembly when viewed from a top view. It is connected directly to the rotor shaft and is identified as the driving gear. The second gear is fixed at its centerline to the housings such that it intermeshes with the driving gear.

The gears are placed within a closely fitting cavity that resembles a figure eight. There is an inlet path and an outlet path at the coincidence of the figure eight formed by two circles (cylindrical shapes).

Liquid is sucked in at the inlet side of the pump and carried within the gear cavities as the motor rotor rotates the driving gear. The liquid contained within the gear teeth cavities is forced out of the outlet side of the pump.

The gear housing assembly is sealed by a peripheral “O” ring against an intermediate plate so there cannot be leakage. Similarly, an “O” ring on the opposite side of the plate seals the stator portion of the motor.

This design eliminates the need for a magnetic clutch or, alternatively, dynamic shaft seals.

Batteries to power the EMT conditioning unit may be held in a battery tray. The battery tray may be located, for example, at the top of the housing of the EMT conditioning unit.

Filters may be used in the coolant pathways, as for example, filter 1312 (FIG. 14) and filter 1412 (FIG. 15). Filter 1312 of the EMT unit may be positioned in the bottom of the housing of the unit 13. In the ICU unit 19, filter 1412 may be positioned on the outlet line from reservoir 1411.

A filter assembly 1347 (FIG. 14) may be used in the auto-fill kit. A fill kit filter has a serviceable removable filter element. When the EMT is run in the auto-fill mode, the internal filter is back-flushed. The fill kit filter is used as a debris trap and can be removed, back flushed or replaced. This filter may also be used in the ICU Auto-Fill Kit.

The use of a replaceable or back flushable filter in the Auto-Fill kit eliminates the need for quick disconnects in either the ICU or EMT units and simplifies the assembly and serviceability of either system.

The cooling process may be commanded to start by a program that is executed after the system's power-on self-test (POST) is complete, but only if the system has passed all of the POST tests.

Referring again to FIG. 3B, the collar 44 may have a Velcro hook material along its sides for connection to strap 46. Further, collar 44 may be constructed from a back portion 48 and two side members 50. Each side member 50 is hinged to back member 48 by interlocking knuckles with a cylindrical void in common alignment with each. A hinge pin shaft fits snugly within the cylindrical void of each knuckle so as to connect a side member 50 to the back portion 48 while allowing rotational movement of side member 50 about the hinge pin axis. An expandable tubing, e.g., Tygon tubing may surround the shaft to provide friction to the movement of a side member relative to the back member. An elastomeric spring may be placed at either or both ends of the hinge to create a frictional hold. Between (1) the user's neck and (2) the side members and back member, a breathable medical foam layer may be positioned. The extent of rotation of the side member relative to the back member may be established. Further, the side member may be shaped so as to provide comfort along the edge of the side member and to permit ease of handling via its far end.

1. A system for cooling a human body, comprising:
a source of liquid heat transfer coolant;
a cooling cartridge heat sink;
a heat exchanger for attachment directly to the human body;
a temperature probe connectable to the human body for generating body temperature data;
set point temperature data;
a fluid pathway located between said source and said heat exchanger, said pathway including a first path and a second path, said first path disposed in proximity to said cooling cartridge so as to cool said heat transfer coolant flowing along said first path, said second path bypassing said cooling cartridge so as to avoid cooling of said heat transfer coolant by said cooling cartridge; and
a controller configured to control the flow of said heat transfer coolant along said first path and said second path as said heat transfer coolant passes to said heat exchanger, said controller configured to use said body temperature data and said set point temperature data in control of said flow.

2. A system according to claim 1 wherein said fluid pathway includes a first route connecting said source to said heat exchanger for transfer of said coolant from said source to said heat exchanger; and a second route connecting said heat exchanger to said source for transfer coolant from said heat exchanger to said source.

3. A system according to claim 2 wherein said second route includes said first path and said second path.

4. A system according to claim 3 and further including a control valve located in said fluid pathway, said control valve being responsive to said controller to direct said coolant relative to said first path and said second path.

5. A system according to claim 4 wherein said control valve includes two valve positions: an OPEN position and a CLOSED position, said control valve being controllable to one of said two positions by said controller.

6. A system according to claim 5 wherein said valve directs coolant along said first path to said source when said control valve is in said CLOSED position, and directs coolant along said second path to said source when said control valve is in said OPEN position.

7. A system according to claim 4 wherein said control valve is actuable by a digital signal.

8. A system according to claim 1 wherein said set point temperature data includes a plurality of set points, each one of said set points associated with a temperature.

9. A system according to claim 8 and further including a time counter configured to provide time output data.

10. A system according to claim 9 wherein said controller is configured to utilize said time output data in control of said flow.

11. A system according to claim 1 wherein said set point temperature data includes a final set point datum associated with a final temperature.

12. A system according to claim 11 and further including a user interface, said user interface configured to receive user input of said final set point datum.

13. A system according to claim 1 and further including a housing containing (1) said source of heat transfer coolant, (2) said cooling cartridge and (3) said controller.

14. A system according to claim 13 and further including a tubing defining a portion of said pathway, said tubing disposed between said housing and said heat exchanger.

15. A system according to claim 1 and further including a cooling heat exchanger disposed in proximity of said cooling cartridge, said cooling heat exchanger defining at least a portion of said first path and configured to cool said heat transfer coolant via said cooling cartridge.

16. A system according to claim 1 and further including an air pump, said air pump connected to said heat exchanger.

17. A system according to claim 16 and further including an air pathway located between said air pump and said heat exchanger; and a tubing defining a portion of said pathway and a portion of said air pathway.

18. A system according to claim 15 and further including an air pump, said air pump connected to said cooling heat exchanger and connected to said heat exchanger.

19. A system according to claim 13 wherein said cooling cartridge is removably mounted to said housing.

20. A system according to claim 1, and further including a cooling heat exchanger disposed in said first path so as to receive said liquid heat transfer coolant, said cooling heat exchanger being cooperatively coupled to said cooling cartridge so as to cool said heat transfer coolant.

21. A system according to claim 20 and further including an air pump connected to said heat exchanger and said cooling heat exchanger.

22. A system according to claim 13 and further including an air pump connected to said heat exchanged, said air pump being contained within said housing.

23. A system according to claim 22 and further including a tubing defining a portion of said pathway, said tubing disposed between said housing and said heat exchanger.

24. A system according to claim 23 and further including an air pathway located between said air pump and said heat exchanger; and a tubing defining a portion of said pathway and a portion of said air pathway.

25. A system for cooling a human body, comprising:
a source of liquid heat transfer coolant;
a cooling cartridge heat sink;
a heat exchanger for attachment directly to the human body;
a fluid pathway located between said source and said heat exchanger, said pathway including a first path and a second path, said first path disposed in proximity to said cooling cartridge so as to cool said heat transfer coolant flowing along said first path, said second path bypassing said cooling cartridge so as to avoid cooling of said heat transfer coolant by said cooling cartridge;
a control valve located in said fluid pathway relative to said first path and said second path; and
a manual control interface cooperatively coupled to said control valve to control the flow of said heat transfer coolant along said first path and said second path as said heat transfer coolant passes from said heat exchanger to said source.

26. A system according to claim 25 wherein said manual control interface includes a control knob.

27. A system according to claim 26 wherein said control knob is rotatable through a range representing a linear temperature range.

28. A system according to claim 25 and further including a cooling heat exchanger disposed in proximity of said cooling cartridge, said cooling heat exchanger defining at least a portion of said first path and configured to cool said heat transfer coolant via said cooling cartridge.

29. A system according to claim 25 and further including a housing containing (1) said source of heat transfer coolant, (2) said cooling cartridge, (3) said control valve and (4) said cooling heat exchanger.

30. A system according to claim 29 and further including a tubing defining a portion of said pathway, said tubing disposed between said housing and said heat exchanger.

31. A system according to claim 28 wherein said manual control knob is located on the outside surface of said housing.

32. A system according to claim 25 and further including an air pump connected to said heat exchanger, said air pump being contained within said housing.
